(19) United States
(12) Patent Application Publication
(10) Pub. No.: US 2025/0248592 A1
(43) Pub. Date: Aug. 7, 2025

(54) Visual Intraoral Dental Examination Device, Method, System and Kit

(71) Applicant: Hunte Innovations, LLC, Daytona Beach, FL (US)

(72) Inventor: Liam Hunte, Daytona Beach, FL (US)

(21) Appl. No.: 19/043,320

(22) Filed: Jan. 31, 2025

Related U.S. Application Data
(60) Provisional application No. 63/627,857, filed on Feb. 1, 2024.

(51) Int. Cl. A61B 1/24; A61C 19/04; G16H 40/63; G16H 50/30

(52) U.S. Cl. CPC A61B 1/24; A61C 19/043; G16H 40/63; G16H 50/30

(57) ABSTRACT

The disclosure presented herein describes a novel, useful and non-obvious tool for dental practitioners to multi-dimensionally view, examine, probe, analyze and share all of the foregoing with a patient in real time. The invention embodies a wireless, integrated probe and mirror system in a single electronic apparatus which allows a practitioner, as well as a patient, to have a simultaneous live visual of the mouth throughout any oral procedure. Additionally, the system includes sensors and data collection and transmission components capable of assessing, recording, and storing data for clinical diagnosis and training purposes. Integration of the probe and mirror system into one electronic device establishes an all-in-one system that enables any practitioner to free their hand that is traditionally dedicated to the mirror and have a live visual of the mouth available to them and the patient throughout any procedure.

### CROSS-REFERENCE TO RELATED APPLICATIONS

The present application is related to co-pending U.S. provisional patent application Ser. No. 63/627,857, filed on Feb. 1, 2024 by the same inventor as the present application, Liam Hunte.

### STATEMENT REGARDING FEDERALLY SPONSORED RESEARCH/DEVELOPMENT

Not applicable.

### REFERENCE TO SEQUENCE LISTING, A TABLE, OR COMPUTER PROGRAM LISTING COMPACT DISC APPENDIX

Not applicable.

### BACKGROUND OF THE INVENTION

This invention relates to instruments for performing medical examinations of the interior of body cavities; remote monitoring of patients; illuminating arrangements for the mouth; transmission of vital signals via a communication network; detecting, measuring or recording by applying mechanical forces; evaluating teeth; devices using data or image processing for diagnoses; and probes for depth measuring of periodontal pockets. Specifically, it discloses a novel, non-obvious and useful dental tool capable of multi-dimensional visualization, examination, scanning, diagnostics, and advanced patient interactions with practitioners.

Dentists are trained to use a periodontal probe and mirror together when performing evaluations and treatment of patients. These two dental instruments are a part of the dental toolbox and are essential instruments to dentists to analyze and diagnose the mouth. These tools require the dentist to use both hands to manipulate the angle and position of the mirror in tandem with the probe in order to obtain visual access to areas within the mouth, including the tooth sulcus and the periodontal pocket. Visual access is necessary for proper evaluation and assessment of the treatment area, measurement of periodontal pockets to assess level of bone loss and or inflammation, and early detection of certain oral diseases and tooth decay. The probe serves as the eye of the practitioner beneath the gumline, and the mirror enables visualization around the backs and sides of teeth, pockets and tissue. Presently, new technology has been incorporated into handheld periodontal probes capable of functionality including temperature, pH and depth measurements. However, these instruments do not take advantage of emerging science that includes cutting edge sensing tools, machine learning and advanced visualization systems.

One technological gap lies with the lack of visualization for both the doctor and patient during the examination and evaluation process. Presently, periodontic disease identification involves tactile examination, radiographs, and probing using visual cues for loss of tissue and inflammation. Instruments that are currently available to dental practitioners beyond the probe and mirror have focused on improving accuracy of probing depth; probing accuracy depends on practitioner experience and early signs of periodontic disease can often be missed. Equipment used in typical oral examination includes endoscopic cameras of various configurations; some feature a light that is absent of any tool allowing for the doctor to operate simultaneously. As mentioned, probes with a built-in thermistor and pH meter to collect temperature and acidity data to be communicated using Bluetooth have also been described. But these devices still fall short of providing both the practitioner and the patient quantifiable information for early disease detection and fail to enable views of the interior of the mouth, teeth, and tissue accurately and clearly. Limitations in current methodology include fogging or blocking of the mirror, difficulty with viewing and recording present-state patient condition, and the need to engage both hands to separately hold the probe and the mirror. Also, it would be advantageous in some situations for the patient to be able to view what the practitioner is doing and seeing; the probe and mirror is not effective for this purpose.

Another shortfall in presently disclosed or commercialized systems that use temperature sensing is with precision and accuracy. Currently available systems typically utilize a thermocouple or resistive temperature detector (RTD) connected to a probe tip for temperature sensing. The probe tip must come in contact with the patient's gum and can only analyze one section of the mouth at a time. Probes using this type of thermal sensing are therefore functionally limited. In contrast, the present invention provides a system to canvas the entire mouth at once by using non-invasive thermography, also known as infrared thermal imaging, and incorporates one or more infrared cameras.

In-office measurement of patient biomarkers can reduce time and errors. However, most current patient biometric tests utilize colorimetric detection, or measure only a single biomarker. The present system is capable of simultaneous detection and capture of multiple substantial biometric measurements and sharing them in real time with both patient and doctor.

The disclosure presented herein describes a novel, useful and non-obvious tool for dental practitioners to multi-dimensionally view, examine, probe, analyze, generate multi-dimensional models, and share all of the foregoing with the patient in real time. Furthermore, it is operational with one hand, such that a dental practitioner or user can use the other hand to perform dental treatment using other tools; integration of the probe and mirror system into one electronic device establishes an all-in-one system that enables any dentist to free their hand that is dedicated to the mirror and have a live visual of the mouth available to them and the patient throughout any procedure.

### BRIEF SUMMARY

The invention presented comprises an apparatus; a method of using the apparatus; a system integrating the apparatus, method and a computer assisted process for visually sharing the apparatus output; and a kit further comprising the various components of the system such that they can be easily and safely stored and transported. The invention embodies a wireless, integrated probe and mirror system in a single electronic device which allows a user, as well as a patient, to have a live visual of the mouth in real time throughout any oral procedure, while only requiring a single hand from the user. Additionally, the system includes sensors, data collection, and transmission components coupled with goggles and auxiliary computer interfacing adapters, that wirelessly communicate with the probe. The auxiliary adapters are designed to operate with a custom multi-platform (mobile or desktop) software application for added patient health insights, patient specific information management, tele-conferencing, and multi-dimensional model generation by using all the data wirelessly transmitted by the probe. So, the system is capable of assessing, recording, storing and visualizing data for clinical diagnosis, visualization by a patient, and training purposes. The incorporation of a plurality of cameras, thermal sensors, and probes, along with a number of other elements that significantly expand functionality of a traditional dental probe, provide dental professionals and their patients with a considerably enhanced clinical experience.

Generally, improvements to the present art include allowing a dental professional to use a single hand for what in the past required two, as well as more efficient integration of viewing methods of the oral condition for both the user and the patient. As a tool to be used in combination with radiographic findings (X-Rays), this tool will generate a greater understanding of the specific oral condition of a patient. This technological advancement of the probe and mirror has the potential to revolutionize the dental profession because it can enhance communication between the doctor and the patient and possibly lead to greater treatment acceptance.

More specific advancements of the present invention include the ability to generate actual landscapes of the interior of the mouth, as opposed to present digital cameras which just generate models which approximate the interior landscape. The present invention utilizes visible and infrared light spectrum energy sensing technology to build real images. When an infrared camera is focused on the inside of the mouth, for instance the bottom set of teeth, temperature variance is visually detailed based on the infrared signature of all matter in view of the camera. An infrared sensor module is incorporated into the device and system to facilitate thermography. In this way, the invention is able to canvas the entire mouth at once by using non-invasive thermography, also known as infrared thermal imaging. Utilizing computer assisted processes, the thermal imagery and visible light imagery is wirelessly transmitted from the probe to an integrated virtual or augmented reality (AR) goggles on command, displaying the full view of the mouth and any localized high or low temperatures. Hence, the probe creates a composite view of the mouth which encapsulates the information from the infrared and visible light data channels which can be viewed as one image frame, or separate channels. Further, the probe's software can categorize any localized elevated temperatures as anomalies using a deterministic logical algorithm and notify the practitioner. Now, the pursuant channels described are two of four frames that are captured, processed, and wirelessly transmitted by the probe in real-time. The probe features stereo vision by using forward facing visible light sensors stationed aft the probe head, near the doctor's hand, to capture an outside wide field-of-view look of the patients mouth for situational awareness, and doctor and patient physical orientation perception. Therefore, the probe not only creates two channels, an infrared and visible light image frame for closeup non-invasive visual inspection of any region inside the oral cavity, or a multi-position scan from outside or inside the cavity, but also two additional visual channels for added treatment awareness. Both camera arrays that foster these channels incorporate image signal processing (ISP), electronic image stabilization (EIS) and electronic zoom via embedded software on a microprocessor unit (MPU).

A biopotential sensor, load cell sensor, Time-of-Flight (ToF) sensor, accelerometer, and a gyroscope are positioned near the interchangeable probe tip mount mechanism. By heavy gold plating outside sections of the probe tip, the tip is transformed into a two terminal-electrode necessary for biopotential sensing, and via internal conductive traces, the probe tip also functions as a thermistor for thermal sensing. This sensor suite, in conjunction with the near field of view infrared and visible light camera array, are used for pocket depth determination, doctor awareness of applied force to a periodontal pocket or gingival sulcus, and oral temperature perception. Further, at least three levels of redundancy are employed for pocket depth estimation: machine-vision analysis of graduated probe tip markers, machine vision depth analysis, probe-tip electrical resistance-to-depth relationship, and ToF distance estimation. For gingival sulcus depth analysis, one level of redundancy is employed: machine-vision analysis of graduated probe tip markers, probe-tip electrical resistance-to-depth relationship.

To reiterate, the system described encapsulates at least four different forms of non-destructive testing: automated optical inspection, microscopic inspection, remote visual inspection, and thermography. Automated optical inspection is a part of the series of embedded machine-learning models that utilize sensor fusion to enable device-orientation recognition, speech-command recognition, periodontal probe depth determination, oral-cavity health classification, oral-cavity image segmentation and oral-cavity depth analysis on the microprocessor. The embedded machine-vision neural network models ingest and fuse data from the image sensors and extracts important spatial features. All of this data is integral to the practitioner to generate inferences and prognoses of patient health conditions and to ascertain the safety of conducting a particular procedure at the desired oral region.

Oral cavity multi-dimensional model creation is enabled using photogrammetry software on the mobile application. An example of this workflow would include the following steps: connect auxiliary-adapter to computer, engage mobile-side software application, engage probe-side photogrammetry mode using speech command, center the probe-head (without probe-tip) with respect to the anterior oral cavity, begin overlapping picture capture around mouth, end image capture, view post-processed result (multi-dimensional model) in the mobile-application.

Similar to related art, the present invention features a thermal sensing probe tip. However, this invention is distinguished in that the thermal sensing probe tip has accessory functions as part of the probe's sensor suite. The purpose of the thermal sensing probe tip of the present invention is to provide changeable functionality to enable the practitioner to conduct non-invasive inspection of an anomalous oral region. This investigation can lead to the discovery of dead tissue, necrosis, bone loss or other infection that can impact the success of, or prohibit treatment.

A further feature of the present invention includes methodology and components that enable the use of a battery hot-swap system. As an emerging Medical Artificial Intelligence of Things (MAIoT) technology, this invention includes optimized powering components and the ability to change out batteries during use without losing information or time. A hot-swap subsystem is configured such that when the main battery returns after being disconnected, it is prioritized. Until the battery returns, a supercapacitor provides power to the circuit for a set time limit. Upon the batteries return above a set voltage potential, the supercapacitor is charged from the battery. If an optional wired power source is connected, that source is prioritized thereby simultaneously supplying power to the battery, supercapacitor, and the remaining circuit.

It is an object of this invention to provide dental practitioners with an apparatus capable of assessing, probing, evaluating and visualizing intraoral cavities with just one hand.

It is a separate object of this invention to provide dental practitioners with a methodology to visually share their findings with their patients multi-dimensionally, in real time.

It is another object of this invention to provide a dental tool capable of automatic measurement, data capture, sensing and information storage and processing capabilities.

It is a further object of this invention to provide visual and biometric information to a practitioner and a patient simultaneously using advanced personal protective equipment capable of display and data sharing.

It is a different separate object to reduce the physical armamentarium of the dentist, while increasing their virtual awareness.

### REFERENCE CHARACTERS USED IN THE DRAWINGS

1. probe neck enclosure
2. probe handle enclosure
3. probe HotSwap enclosure
4. probe head cover
5. probe tip
6. right angle prism
7. achromatic doublet lens
8. rod lens
9. visible light aspheric lens
10. probe visible-light image sensor
11. Light-emitting diode (LED)
12. probe long-wave infrared image sensor
13. probe temperature sensor
14. probe PCB (front, middle, antenna, battery)
15. Inertial Measurement Unit (IMU) (6-axis/9-axis)
16. Wi-Fi/Bluetooth/GNSS/eSIM Transceiver(s)
17. 2.4 GHz/5.8 GHz Transceiver/RF front-end
18. Power delivery PCB
19. infrared aspheric lens
20. primary battery
21. Time-of-Flight sensor
22. Load cell sensor
23. Biopotential sensor
24. Grub screws
25. uninterruptible power supply (UPS)/Supercapacitor
26. Wide Field of View stereo camera light path
27. Infrared optical arrangement
28. Biopotential electrode
29. Board-to-board PCB connector
30. grasping surface
31. Stringer
32. Phase-change material (PCM) passive thermal management
33. Universal Serial Bus (USB) interface connector w/Display-port
34. Mu Foil (Shielding)
35. Magnet
36. O-ring (Nitrile)
37. probe tip fastener
38. battery cover
39. probe interconnect
40. PPE/AR goggles
41. Pogo-pins (gold)
42. eSIM (SIM card)
43. Magnetometer
44. Microcontroller unit (MCU) multi-protocol transceiver (3 GHz/LTE/Wi-Fi/Bluetooth/GNSS/NFC)/RF front-end
45. Temple piece
46. Momentary button
47. Neural processing unit (NPU) low-power
48. FPGA
49. Proximity Sensor
50. Temple-to-bridge metal hinge
51. LED Driver
52. RGB-LED Driver
55. Ear rest
65. Polarized layered combiner lens (wide FOV)
66. RGB-:ED
67. Charging hub PCB
68 Charging hub enclosure
69. Wall outlet connector
70. Embedded rechargeable battery protrusion (right and left)
75. Head-band
76. OLED micro-display panel
77. PPE/AR goggle PCB
78. Microphone
79. Power management system (PMS)
80. Central-processing unit (CPU)
81. RAM memory
82. Flash storage
83. Power management system (PMS) w/HotSwap
84. Display Convertor
86. MicroSD (Secure Digital) card slot
87. High-Definition Multimedia Interface (HDMI) connector
89. Interface cable
90. Monitor
91. Desktop computer
92. Internet access via Ethernet or Wireless local area network (WLAN) Wi-Fi router
93. Data/Image
94. Wireless Peer-to-Peer (P2P) network via 2.4/5.8 Ghz RF front end
95. Computer/display auxiliary adapter dongle: 2.4/5.8 Ghz receiver (optional)
96. Patient's mouth
97. Desktop/Mobile VIDE software application
98. Dongle
99. Antenna
100. Practitioner

### BRIEF DESCRIPTION OF THE SEVERAL VIEWS OF THE DRAWING

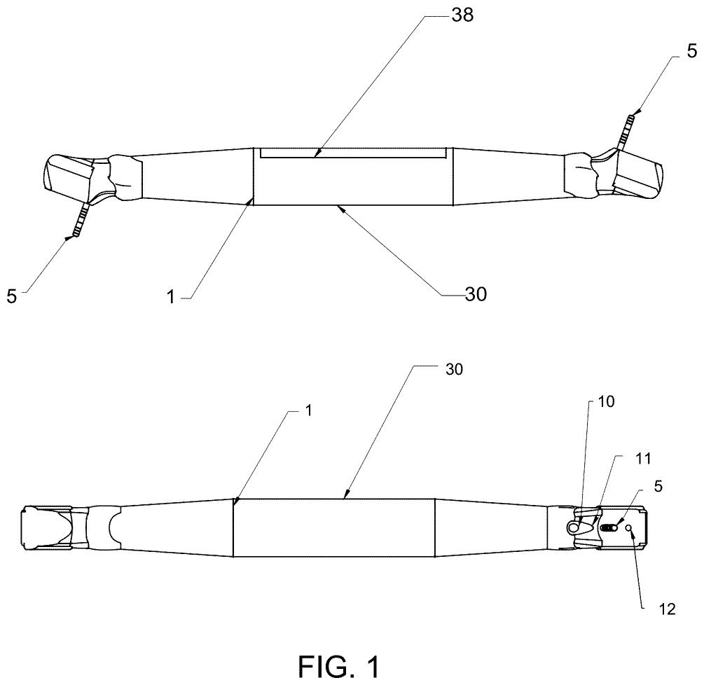

FIG. 1 is a perspective and an elevation view of an embodiment of the probe apparatus of the invention.

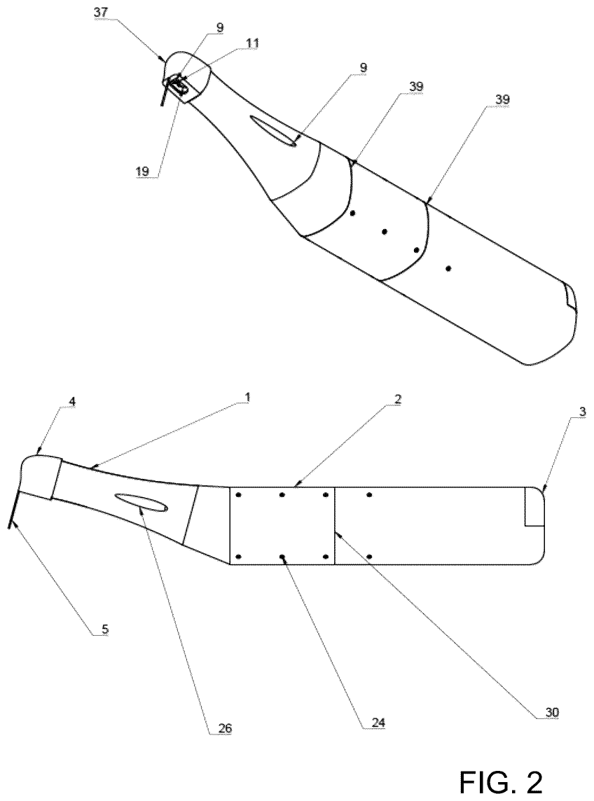

FIG. 2 shows a perspective and an elevation view of an alternate embodiment of the probe apparatus shown in FIG. 1.

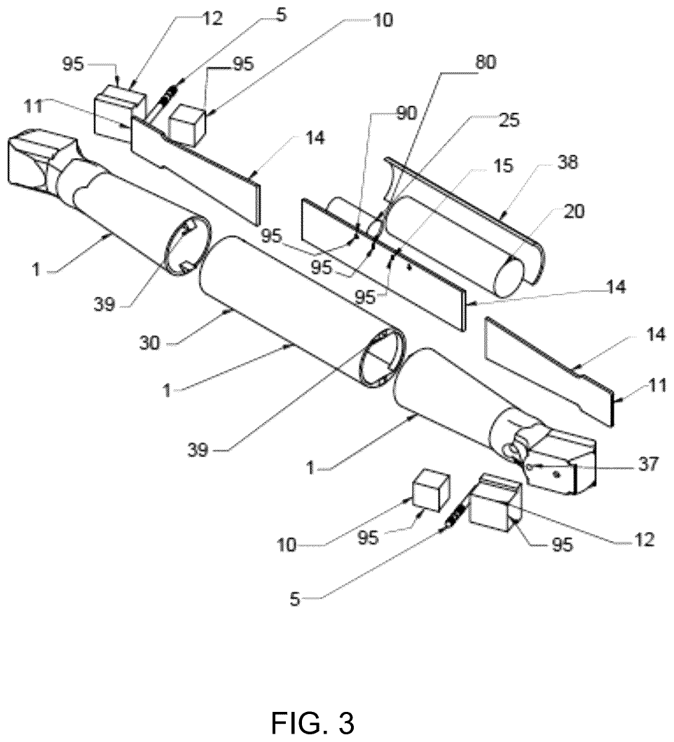

FIG. 3 is an exploded perspective view of an embodiment of the probe apparatus shown in FIG. 1.

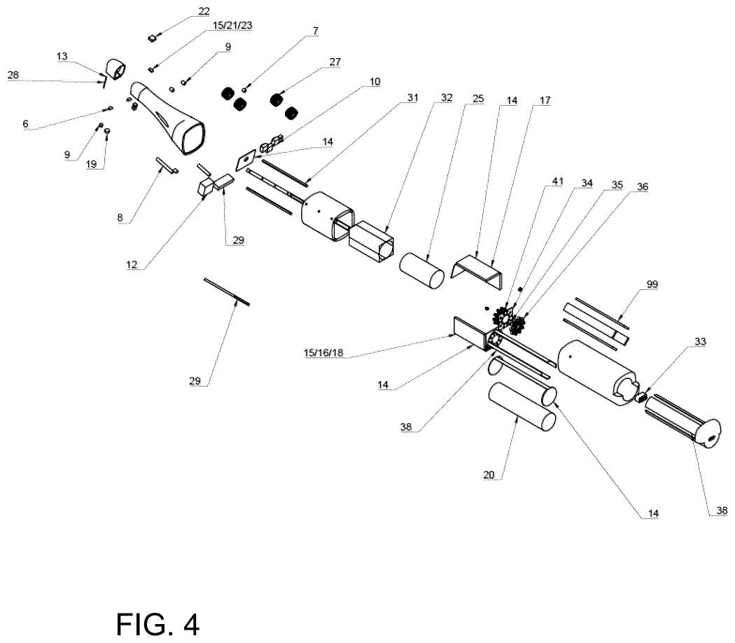

FIG. 4 is an exploded perspective view of an embodiment of the probe apparatus shown in FIG. 2.

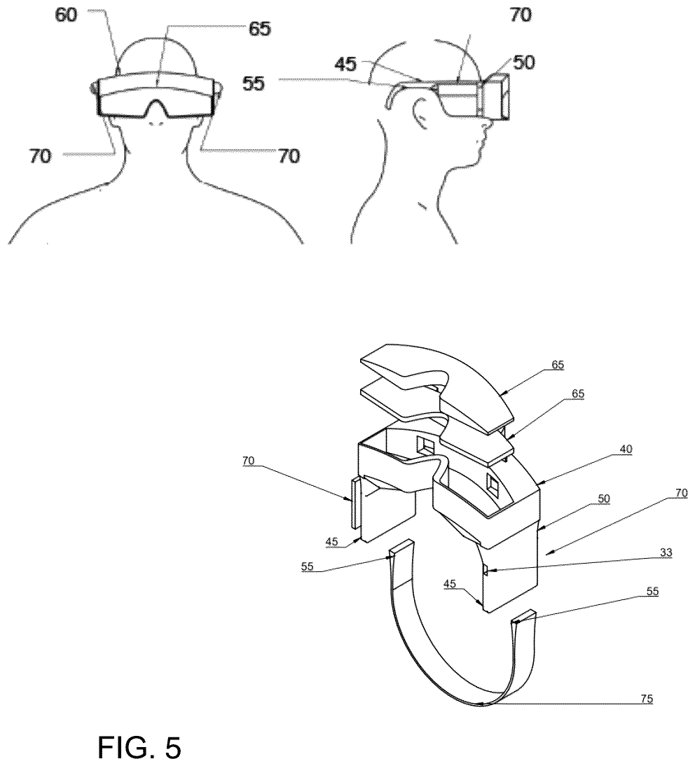

FIG. 5 is a schematic representation of the PPE/AR goggles as worn by the practitioner in a front and a side view, and an exploded view showing parts of an embodiment of the PPE/AR goggles.

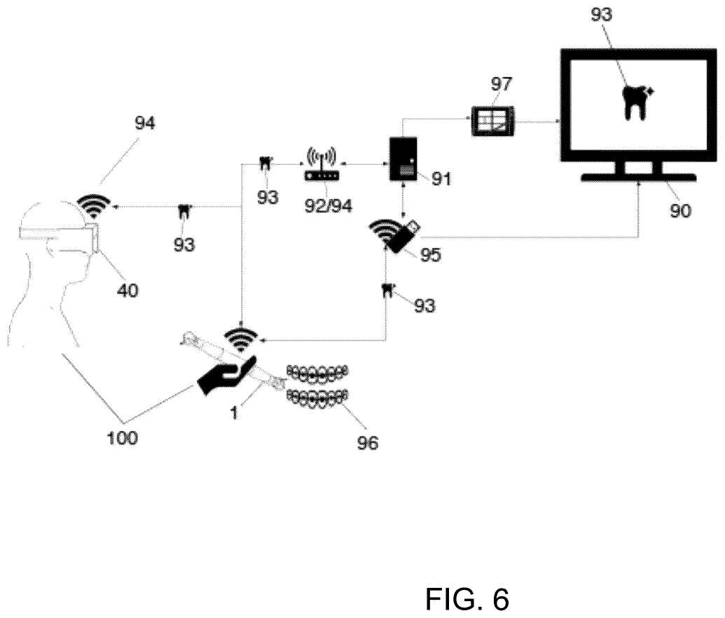

FIG. 6 is a schematic flow diagram showing the interconnected components of the of the invention comprising the system, including the wirelessly connected probe apparatus, PPE/AR goggles, and computer assisted processing hardware/software.

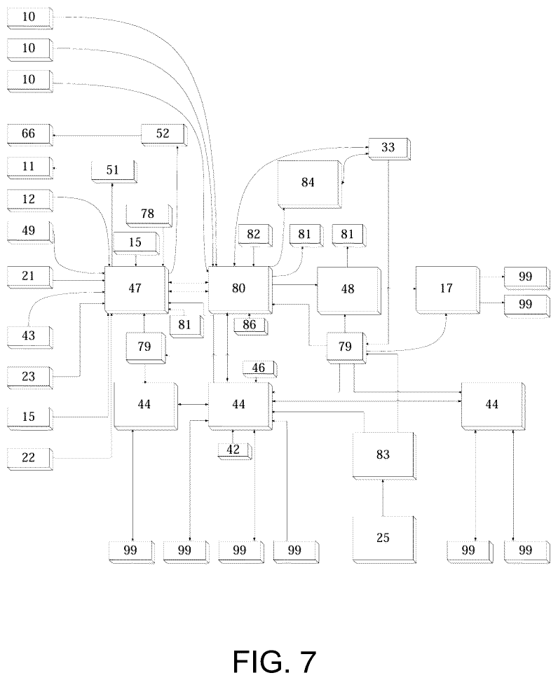

FIG. 7 is a block diagram illustrating the interconnected components operably deploying the computer assisted processes of the probe apparatus of the invention.

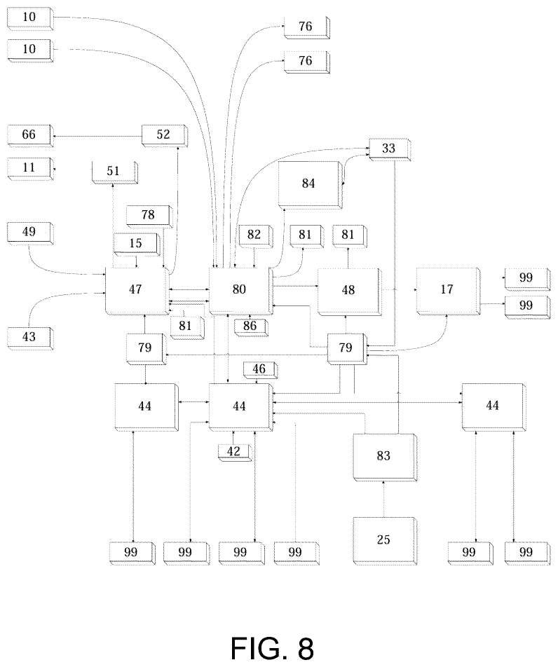

FIG. 8 is a block diagram illustrating the interconnected components operably deploying the computer assisted processes of the PPE/AR goggle apparatus of the invention.

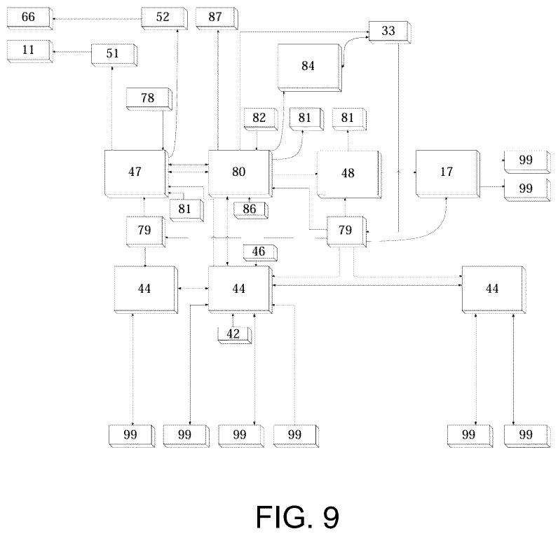

FIG. 9 is a block diagram illustrating the interconnected components operably deploying the computer assisted processes of the auxiliary adapter of the invention.

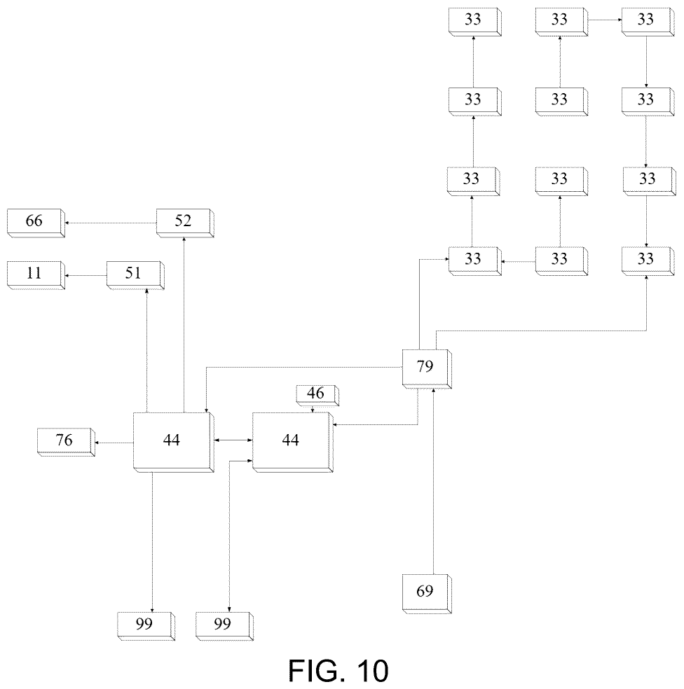

FIG. 10 is a block diagram illustrating the interconnected components operably deploying the computer assisted processes of the charger apparatus of the invention.

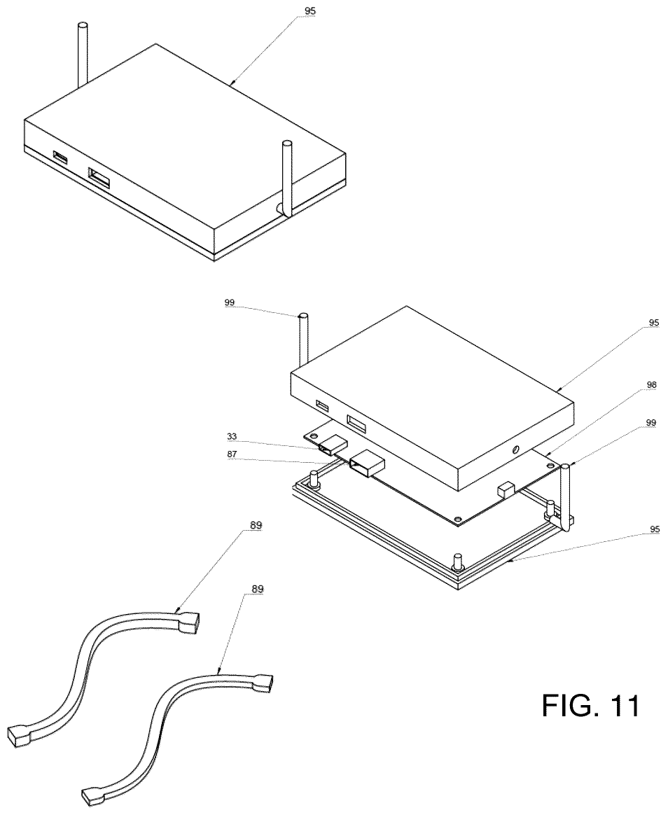

FIG. 11 is a perspective and an elevation view of an embodiment of the auxiliary adapter apparatus of the invention.

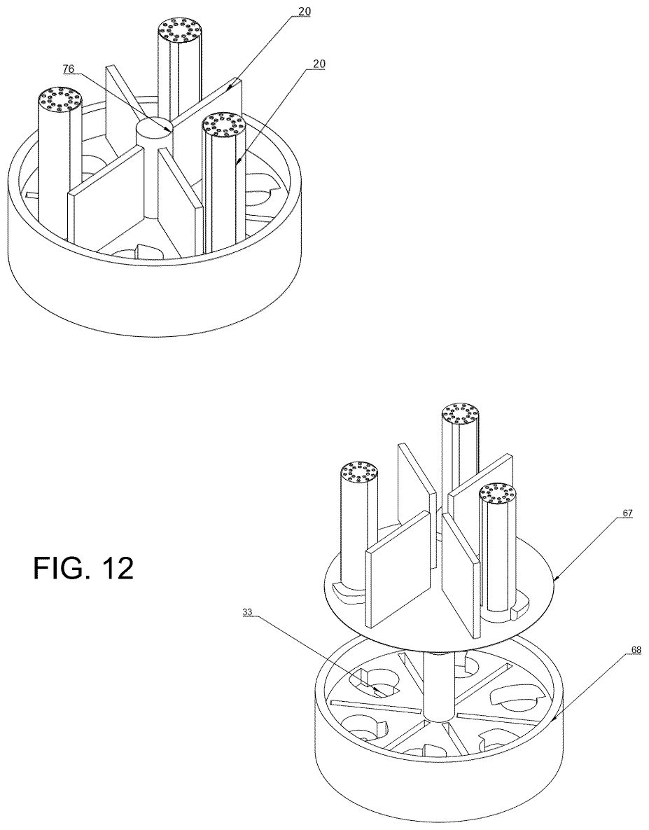

FIG. 12 is a perspective and an elevation view of an embodiment of the charger apparatus of the invention.

### DESCRIPTION OF THE INVENTION

While various embodiments are described herein, it should be appreciated that the present invention encompasses many inventive concepts that may be embodied in a wide variety of contexts. Illustrative embodiments of the invention are described below. Not all features of an actual implementation for all embodiments are necessarily described in this specification. In the development of any such actual embodiment, implementation specific decisions may be made to achieve the design specific goals, which may vary from one implementation to another. It will be appreciated that such a development effort would be a routine undertaking for persons of ordinary skill in the art having the benefit of this disclosure.

The device and method detailed in the following description and drawings provide a novel, non-obvious and useful dental tool that advances both practitioner and patient dental experience. The invention disclosed comprises an AR/VR experience for the practitioner and projects what the practitioner “sees” for the patient in real time onto any USB3.0, HDMI, or DisplayPort compatible display.

FIG. 1 shows both a perspective and an elevation view of an embodiment of the visual intraoral dental examination probe apparatus tool of the invention. The apparatus comprises a wireless probe and mirror system integrated into a single electronic probe apparatus that is operable with one hand to enable improved clinical assessment and visualization. This embodiment has a first end and a second end, with a probe tip 5 on each end of the probe apparatus. The primary electronic function of the probe apparatus is to transmit video data to the PPE/AR goggles 40, which is a head-mounted display system comprising personal protective glasses transformed to possess a ‘heads up display’ allowing for visual access to the patient's mouth using video data captured by the one or more cameras of the system (PPE/AR goggles 40 and cameras are not shown in this figure). One or more cameras may be configured to provide real-time visualization of the oral cavity. The cameras are operatively connected with a sensor suite, positioned proximal to and are operatively connected with an interchangeable probe tip 5, which includes one or more sensors, which may include but are not limited to probe visible-light image sensors 10, probe long-wave infrared image sensors 12, Time-of-Flight (ToF) sensors 21, load cell sensors 22, and biopotential sensors 23. Functionality may be visualized by light-emitting diodes (LED) 11. In a preferred embodiment, communication between the tool and PPE/AR goggles 40 is facilitated using the VSA network, which is comprised of a ultra-low latency high-data throughput 2.4/5.8 Ghz software-defined radio and a separate ultra-low latency low-data throughput 3 Ghz software-defined radio. Interaction in both virtual reality (VR) and alternate reality (AR) environments are enabled and contemplated by the invention. One implementation grants the user the option of two viewing modes to avoid the concerns of motion sickness associated with AR. In one embodiment, the two separate modes that are easily switched between due to a motorized optical lens arrangement include alternate birdbath-image projection mode (BIPM) and horizontal-split screen mode (HSSM). Another implementation possesses only a single AR viewing mode which is enabled by optical wave guides built into the main lens enabling simultaneous view of two light sources in a more compact arrangement that fosters device weight reduction. Importantly, this system does not require Wi-Fi to transmit video; the system creates, manages and upholds its own private IoT network that operates in the ISM band 2.4/5.8 Ghz, through signal generation and frequency modulation by a baseband processor to an analog-to-digital converter RF front end designed to transmit in the particular ISM frequency range within the regulated RF transmission energy levels. The embodiment of the system described features coprocessors that manage, NFC quick-pair, eSIM text-messaging, GNSS tracking, Wi-Fi cloud storage connectivity and tracking, Bluetooth pairing and tracking, and private IoT transmission band communication, and awareness of all similar devices in its vicinity. The ability to detect other similar related and connected devices outside the current private network instance, is called VIDE Situational Awareness (VSA). This program grants the probe to ditch frequency bands to avoid interference from any other VIDE network in its range.

The probe apparatus is also capable of biometric sensing and data capture, using probe tips 5, which may be positioned upon the probe apparatus using one or more probe tip fasteners 37. Since the tool features interchangeable metal probe tips 5, one or more probe tip fasteners are used to releasably attach probe tips 5 on each end of the tool. This feature allows for different graduated probe tips 5 to be used with one probe apparatus, another novel capability of the system. In a preferred embodiment, a safe ratio between the length and end point diameter, per ANSI Standards of 0.4-0.6 millimeters, is used to retain the strength of the probe tip 5; in various embodiments the probe may be longer or shorter to accommodate custom configurations. The probe apparatus may further comprise a gyroscope and accelerometer positioned near the probe tip 5 and in operational connection with the probe tip 5 for detecting the orientation and movement of the probe apparatus during use.

According to American National Standards Institute (ANSI) dentistry standards, hand tools, including probes, must be strong and chemically resistant to allow for reliable usage during treatment when force is applied to the tool, and reliable sterilization after treatment. Since radio waves must be able to freely pass through the tool, construction of the apparatus requires a suitable material that is not opaque to radio waves like all conducting metals. Therefore, in a preferred embodiment, the probe tips 5 and exterior components of the probe apparatus including the probe handle enclosure 2, which surrounds the exterior of the probe apparatus where it is grasped at the grasping surface 30, are constructed out of material with properties including a high yield strength and chemical resistance. In such an embodiment, composite material or autoclave safe material capable of maintaining most, if not all of the material's tensile strength after a high number of sterilization duty cycles, such as polyetherimide plastic, would be used. The tool is comprised of material capable of electrical insulation, moisture isolation, radio-wave transparency, chemical sterilization, heat dissipation (heat from camera modules and MPU), heat isolation (ambient and sterilization temperatures). Furthermore, the tool must be light weight and reusable. In one embodiment, polyetherimide is used in construction, as it is a semi-transparent high strength plastic material that can operate in high service temperature environments. It is resistant to hot water and steam and can withstand repeated cycles in a steam autoclave. These materials are exemplary, and not exclusive; it is to be understood that other materials with similar properties could be used and fall within this disclosure. In one embodiment, the device can be sanitized using cold chemical sterilization (CCS) process, which involves cleaning with a neutral pH detergent before immersing the tool in a CCS solution such as Glutarldehyde, Ortho-phthalaldehydes (OPA), Hydrogen peroxide or Peracetic Acid (IEHP, n.d.). Therefore, the device can undergo CCS, and any metal components that do not enclose electronics can be autoclaved, such as the interchangeable probe tip and head cover. However, the interchangeable probe-head cover 4 and the probe neck enclosure 1 must be machined from the same metal as the probe tips 5 to avoid inter-metal interaction that can lead to galvanic corrosion. The rear portion of the body is split into at least two parts, including an antenna enclosure enclosing the antenna 90 and a removeable battery enclosure covered by a battery cover 38; further, it is constructed from a chemically resistant, and low water absorption plastic. This embodiment also allows for the integration of a battery hot-swap system since the battery enclosure is removable. This system would enable the practitioner to use the probe throughout the workday, from procedure-to-procedure, without needing to charge the apparatus. The tool further incorporates a sealed electronic enclosure that is sterilization safe.

In FIG. 2, an alternate embodiment of the probe apparatus is presented. In this embodiment, there is a single probe head on a first side of the apparatus. Because there is only one end incorporating a probe tip 5, more features can be included within the body of the apparatus without adding bulk or weight to it. These features could include, but are not limited to, additional sensors, better wireless capability and expanded viewing, processing and transmission capacity.

Referring now to FIG. 3, a perspective exploded view of the embodiment of the probe apparatus of the invention with multiple probe heads containing probe tips 5 is shown. The probe apparatus's heads are capable of housing both visible light and infrared cameras, which are further capable of independent or concerted real-time visualization. The electronic components of the apparatus are fitted and positioned within one or more sections of the probe apparatus such that they can be interconnected, and may include an IMU, UPS, probe battery and other functional hardware and software. The outside of the probe apparatus comprises a battery cover 38 providing immediate access for power, and a grasping surface 30 to allow the practitioner 100 secure contact with the apparatus. FIG. 3 also shows the relative positions of the probe tip 5, probe visible-light image sensor 10, LED 11, and probe long-wave infrared image sensor 12 in an embodiment of the apparatus. In one embodiment, a software-defined radio is implemented to transmit H.264/H.265 encoded video data. This function is facilitated using a video-processing unit (VPU), FPGA (field-programmable gate array) baseband processor, and an RF front end. An additional radio that operates in a different frequency band is utilized to transmit all other necessary data with real-time-latency.

FIG. 4 is an exploded perspective view of an embodiment of the probe apparatus shown in FIG. 2. As previously described, this embodiment features a single probe head and probe tip 5 on a first side of the apparatus, and allows for more functional components, expanding technical capabilities without compromising tool weight and comfort for the practitioner 100.

FIG. 5 is a drawing of the PPE/AR goggles 40 as worn by the practitioner 100 in a front and a side view, and an exploded view showing parts of an embodiment of the PPE/AR goggles 40. In a preferred embodiment, at least one visual or audiovisual interface in communication with a software application and capable of display of information generated by the probe apparatus in real time is incorporated into the system. L is incorporated into personal protective equipment (PPE) glasses to reduce weight and achieve an ergonomically appealing form factor. These PPE/AR goggles 40 function to protect the practitioner 100 as well as provide a video or audiovisual interface.

The PPE/AR goggles 40 enable control of the probe apparatus, thereby enabling the practitioner 100 to take photos using the probe apparatus during examination and evaluation for treatment documentation, which is currently a novelty. The heads-up display is powered by the projection of an image from a micro in-plane switching (IPS) or organic light-emitting diode OLED micro-display panel 76 onto the interior of the PPE/AR goggles 40 lens. A special film on the interior of the lens facilitates the transition of the image. This display effectuates Augmented Reality (AR).

In a preferred embodiment, low-profile AR glass technology would be incorporated into personal protective equipment (PPE) goggles to reduce weight and achieve an ergonomically appealing form factor. These PPE/AR goggles 40 function to protect the practitioner 100 as well as provide a video interface. A further feature of the PPE/AR goggle 40 equipment is to use the microphone 78 array in the probe apparatus and PPE/AR goggles 40 to translate practitioners' speech for non-English or native-language speaking patients as part of translational science. For instance, the speech translation feature serves to bridge the gap between an English speaker and non-English speaker in the practitioner and patient relationship. This communication feature, which involves speech synthesis (speech-to-text) and translation (text-to-text) of microphone data, works in conjunction with all components of the system.

The PPE/AR goggles 40 enable control of the probe apparatus, thereby enabling the practitioner to take instant photos during examination and evaluation for treatment documentation, which is currently not practicable with presently available technology. The heads-up display is powered by the projection of an image from a micro in-plane switching (IPS) or organic light-emitting diode (OLED) panel onto the interior of the PPE/AR goggles 40 lens. A special film on the interior of the lens facilitates the transition of the image. This display effectuates Augmented Reality (AR).

The system of the invention deploys a software application to perform, process, capture and analyze multiple levels of redundancy for pocket depth estimation, including machine-vision analysis of graduated probe tip markers, machine vision depth analysis, and a probe-tip electrical resistance-to-depth relationship, along with ToF distance estimation. This software, the Desktop/Mobile VIDE software application 97 is a downloadable desktop and mobile application that provides a viewing interface of the procedure from a computing device or a phone. The Desktop/Mobile VIDE software application 97 requires the dongle 98, whereas as for a mobile user wireless connection is established with the probe's peer-to-peer network via a software protocol that leverages the auxiliary Wi-Fi and Bluetooth capabilities built into the dongle 98. Data from the probe apparatus is transferred to the dongle 98 and displayed by the Desktop/Mobile VIDE software application 97. Further, the Desktop/Mobile VIDE software application 97 includes additional features for management of the devices connected to the network.

One or more communication modules or a radio component is incorporated into the system to provide for wirelessly transmitting data to external devices. The computer-display dongle 98 serves as a low-latency wireless connection between a CPU 80 or computer monitor 90 when the system is operative. An adapter is necessary for a computer or standalone monitor 90 to communicate with the VSA network and display data. This component is independent of the probe apparatus and PPE/AR goggles 40, and is not required for those two components to communicate with each other successfully in any mode.

FIG. 6 is a schematic flow diagram showing the interconnected components of the of the invention comprising the system, including the wirelessly connected probe apparatus, PPE/AR goggles 40, and computer assisted processing hardware/software. The components of the system are connected using both hardware as described and depicted and a computer assisted process capable of operably connecting the probe tip 5 and probe apparatus, the PPE/AR goggles 40, the video or audiovisual monitor 90 and the CPU 80. In one embodied method of use, a 5.8 Ghz and 3 Ghz RF (radio frequency) connection between the probe apparatus and the PPE/AR goggles 40 is made, followed by the probe apparatus relaying the data to a project tailored secured app via the dongle that a dental operatory CPU 80 can access and display on a monitor 90 for visual feedback. In a second embodiment of the invention, the probe apparatus sends video data to the PPE/AR goggles 40 and a computer dongle 98 via 5.8 Ghz wireless transmission. Communication between all devices that comprise the system is designed to operate in regions without internet access, which may play a vital role in scenarios of the Doctors Without Borders or similar humanitarian organizations.

One embodiment of the method of using the invention includes, but is not limited to, the steps outlined below. The method for using the invention begins with the device activation process. Initially, the probe apparatus and PPE/AR goggles 40 are turned on by either inserting a charged battery or connecting the device to an outlet using a charging cable and wall charger. Meanwhile, the auxiliary computer adapter is powered via a wall outlet. The device can be “woken up” by pressing a capacitive button or through a low-power speech command, allowing for energy-efficient activation. Following activation, the device pairing step is conducted. This involves pairing the probe apparatus, PPE/AR goggles 40, and adapter through wireless communication. Near Field Communication (NFC) is used to initiate pairing, which then allows for bidirectional peer-to-peer wireless communication through function-specific wireless transmitters, establishing a seamless connection between the devices. The next step involves biometric data acquisition, where the probe apparatus employs a comprehensive sensor suite. This suite includes thermal imagery sensors, visible-light image sensors, biopotential sensors, a load cell sensor, a Time-of-Flight (ToF) sensor, an accelerometer, a magnetometer, a gyroscope, and a microphone. These sensors work in concert to acquire crucial biometric data necessary for assessing the patient's oral health, providing a detailed analysis of the oral cavity's condition. Once the data is captured, the Wireless Data Transmission step ensures that this visual and biometric data is transmitted wirelessly to external devices, including the AR/PPE goggles and the auxiliary computer adapter. This transmission ensures seamless integration with the practitioner's existing digital infrastructure, enabling real-time access to critical information. The data is then subject to data processing and display. The embedded software application on the CPU 80 of the probe apparatus, goggle apparatus, and adapter processes the data to enhance clinical assessments and decision-making.

This processing provides detailed patient health insights, which are crucial for effective diagnosis and treatment planning. For more advanced diagnostics, the method includes multi-dimensional capability including 3D Model Generation. Using Desktop/Mobile VIDE software application 97, which is a desktop software application with photogrammetry technology, multi-dimensional models of the oral cavity are generated. This requires the probe to wirelessly connect to the auxiliary adapter and initialize in photogrammetry mode to capture a series of still images, offering a comprehensive view for accurate diagnosis and treatment planning. The captured data is then subjected to data storage and processing. Information generated by the system is stored and processed on a connected CPU 80 using the internal memory of the probe apparatus, PPE/AR goggles 40, or auxiliary adapter, and through Wi-Fi-based cloud storage services. This supports clinical diagnostics and training, offering a robust platform for ongoing practitioner education and patient record management. Following data processing, communication of results involves sharing patient photos from the probe apparatus or PPE/AR goggles 40 via SMS messaging, leveraging the internal electronic SIM card, or from cloud storage services. Additionally, the desktop software application is integrated into video-conferencing workflows, facilitating teaching and remote practitioner participation. Also, device global positioning is integrated within the method. The probe apparatus and PPE/AR goggles 40 incorporate private GNSS, Bluetooth, and Wi-Fi-based tracking features, enabling a single doctor or an office to locate their devices indefinitely, ensuring device security and availability. In addition, the method may include other enhancements. By way of example and not limitation, the following are additional steps that comprise a method of using the invention: (i). determining pocket depth and applied force: utilizing the sensor suite to determine pocket depth and the force applied to a periodontal pocket or gingival sulcus, providing essential data 93 for dental assessments; (ii). conducting pocket depth estimation; (iii). implementing multiple levels of redundancy for pocket depth estimation, including machine-vision analysis of graduated probe tip markers, machine vision depth analysis, and a probe-tip electrical resistance-to-depth relationship, supplemented by ToF distance estimation, ensuring accuracy and reliability; (iv). displaying processed data on AR goggles in real-time, allowing both the dental practitioner and patient to view the insights simultaneously, enhancing understanding and communication; (v). further generating multi-dimensional models of the oral cavity using photogrammetry software integrated into the software application, and storing and processing captured data on a connected computer through an auxiliary adapter for clinical diagnostics and training purposes; (vi). performing automated optical inspection and oral-cavity health classification using embedded machine-learning models, introducing an advanced layer of diagnostic capability; and (vii). enabling battery hot-swap during procedures without data loss or operational interruption, incorporating a shielded magnetic latching mechanism, battery authentication to prevent tampering, and facilitating on-device or external hub battery charging, ensuring continuous operation and efficiency.

FIG. 7 Visible light (VIS) image sensor 10 hardware is present in the probe and PPE/AR goggles. Infrared light (IR) sensor 12 hardware is present only in the probe. VIS sensors are connected to the CPU 80 via MIPI Camera Serial Interface (CSI). IR sensors are connected to the CPU via Serial Peripheral Interface (SPI). The probe CPU 40 is responsible for processing the raw image data through a sequential pipeline. This pipeline consists of Image Signal Processing (color correction/white-balance) and Electronic Image Stabilization (focusing/frame cropping). If the thermal imagery mode is active, an IR image frame is enhanced by a VIS image frame using an image overlay algorithm to create a single image frame. If the health classification mode is on, then the pipeline is followed by a proprietary domain specific machine learning model before the image frame is encoded in AVC/HEVC format. Also, the CPU 80 has dedicated RAM 81, and fixed 82 and accessible 86 storage. Once encoded, the frame is transferred to the FPGA 48 via high-speed PCIE and Ethernet protocols. The FPGA 48 functions as a baseband processor that generates an analog signal from the CPUs 80 digital output. Further, the FPGA is responsible for forward error correction, modulation/demodulation and constellation formation logic that is necessary for the RF Front End 17. In conjunction with the RF Front End, which is comprised of all necessary circuitry to transmit the signal at a desired center frequency (5.8 Ghz), the video broadcasting unit is formed. Additional coprocessors 44 feature high integration, namely combined processors and wireless modem functionality that enables wireless transmission of lightweight data. Similarly, these components are connected to an antenna 99. One coprocessor 44 manages real-time 3 GHZ communication with all the devices in the IoT network mesh, whereas the remaining two coprocessors manage tasks such as cloud data storage, pairing, telemetry, and tracking. Other sensing components such as 22, 15, 23, 43, 21 49, and 78 connect directly to a neural processing unit 47 for fast computation and inference of intraoral bioinformatics. The proximity sensor 49 enables the device to be aware of probe-tip 5 replacement based on the presence of the probe head cover 4. The battery hot swap 83 implements a supercapacitor 25 to improve the subsystems' robustness by prioritizing long cycle life under high discharge. The microphone 78 primarily functions as an audio source for the small-speech command recognition machine learning model that is executed on the CPU 80. This power management system supplies power to the remaining power management circuits 79 throughout the device. White LEDs 11 serve to illuminate the oral cavity whereas RGB)-LEDs are implemented for user-device customization and visualization of notifications (device mode/battery authentication/speech-command recognition). The primary image source elements in this schematic are the visible 10 and infrared 12 light image sensors that, when combined with lenses, form individual cameras. In the case of the infrared camera, an infrared image sensor is combined with a specific arrangement of optical lenses 19 and mirrors 6 that are optimized for long-wave infrared light transmission within 8000 nm-14000 nm. In the case of the visible light camera, a visible light image sensor is paired with a specific arrangement of 7 optical lenses, light conduits and mirrors to enable light transmission. Optical lenses 9 help to focus and redistribute light-a high-numerical aperture value is preferred for high-resolution, but the values magnitude is impacted by lens size. Optical mirrors and prisms 6, such as gold-protected mirrors enable high-reflectance of light at a desired angle and aid in probe-head compactness. Optical light conduits such as bundled fiber optics or rod lenses 8 enable light transmission in a straight path or flexible transmission around a bent or curved path. The emergency interface 33 is an optional wired connection method between the probe apparatus via the hot-swap enclosure 3 and the PPE/AR goggles 40, or the probe and the auxiliary adapter. The pogo-pins 41 not only act as a conductive terminal between the battery 20 and hot-swap 83 subsystem, but also acts as impedance matched data transmission terminals for the emergency interface 33. This feature encompasses power-delivery and high-speed transmission of large data packets for the doctor to utilize in the case of an emergency, e.g. all-backup batteries 20 are depleted. Structurally, the stringer 31 and grub screws 24 combination help to fasten each part of the probe enclosure together. The phase-change material 32 surrounding the supercapacitor 25 provides passive thermal management within the device. The magnetic shielding foil protects the PCBs 17 99 18 from the parasitic magnetic field of the hot-swap magnet 35. To interconnect different PCBs within the assembly, board-to-board connectors 29 are utilized.

FIG. 8, the principal circuitry inside the PPE/AR goggles 40, operates inversely to FIG. 7's broadcasting unit by receiving the transmitted signal then decoding the AVC/HEVC packet. Once the packet is decoded, the raw image data is available for image pipeline processing, Similar to FIG. 7, the pipeline contains a machine learning stage and frame overlay components. After this stage, one or more micro-displays 76 function as video sinks and show the live video stream. These displays are connected to an image projection path that ends with a polarized combiner lens 65 assembly in the motorized dual-display mode embodiment. Also, the schematic includes image sensors 11 to enable the PPE/AR goggles to possess scene awareness of the of probe in the doctor's hand using machine learning. The microphone 78 primarily functions as an audio source for large-speech command recognition, interaction with a small-language model and speech translation that is on the CPU 80. This apparatus also includes a IoT network mesh coprocessor 44. These internal components are placed strategically, and span throughout the device including the temple piece 45.

FIG. 9 illustrates the auxiliary adapter. This device does not include visible light image sensors 10, infrared image sensors 12, or micro-display panels 76. The broadcasting unit performs to FIG. 8, but the final video sink is an HDMI 87 Tx (transmitter) or an emergency interface 33 for data 93 projection onto a screen. This apparatus also includes a IoT network mesh coprocessor 44. Also, this device does not rely upon a hot-swap battery powered architecture, rather a power-distribution splitter built into downstream power management circuitry 79 of the emergency interface.

FIG. 10 depicts the charging hub circuitry dedicated to battery charge management to support the hot-swap feature. This apparatus also includes an IoT network mesh coprocessor 44. Additionally, a micro-display panel 76 is incorporated as it is necessary for easy display of the charge status of each battery 20.

FIG. 11 illustrates an embodiment of the compute unit that receives data using its exposed antennae 99, and can connect to a computer or drive an external monitor via HDMI 87.

FIG. 12 demonstrates readily available backup batteries 20. Also, this device does not rely upon a hot-swap battery powered architecture, rather power delivery to the power management circuit 79 from a wall outlet connector 69.

The invention further comprises a kit for storing and transporting the invention. The kit includes one or more of the probe apparatus, one or more PPE/AR goggles, replacement components including batteries, probe tips 3, various sensors, a video or audiovisual interface such as a monitor 90, a video or audiovisual monitor adapter, an external data storage drive, and electronic connecting and charging components, all of which are removably positioned in a durable and protective case.

The invention claimed is:

1. An intraoral dental procedures comprising:
a wireless probe and mirror system integrated into a single electronic probe apparatus that is operable with one hand to enable improved clinical assessment and visualization;
a plurality of cameras configured to provide real-time visualization of the oral cavity;
a sensor suite comprising sensors positioned proximal to and are operatively connected with an interchangeable probe tip;
one or more interchangeable probe tips;
one or more communication modules or a radio component for wirelessly transmitting data to external devices;
a software application in communication with the communication module and including a software application interface for processing and displaying data from the probe, and
At least one visual or audiovisual interface in communication with the software application and capable of display of information generated by the apparatus in real time;
wherein the apparatus is configured as a system when connected to and in communication with one or more CPUs.

2. The apparatus according to claim 1, wherein the sensor suite further comprises instructions and components to sense and capture pocket depth determination, doctor awareness of applied force to a periodontal pocket or gingival sulcus, and oral temperature perception, and comprises one or more of the following sensors: thermal sensor, biopotential sensor, probe long-wave infrared image sensor, probe long-wave infrared image sensor, load cell sensor, Time-of-Flight (ToF) sensor.

3. The apparatus according to claim 1, wherein the multi-platform software application includes instructions that enable the system to perform, process, capture and analyze multiple levels of redundancy for pocket depth estimation, including machine-vision analysis of graduated probe tip markers, machine vision depth analysis, and a probe-tip electrical resistance-to-depth relationship, along with ToF distance estimation.

4. The apparatus according to claim 1, further comprising a gyroscope and accelerometer positioned near the probe tip for detecting the orientation and movement of the probe during use.

5. The apparatus according to claim 1, wherein the visual or audiovisual interface further comprises virtual and augmented reality PPE/AR goggles that protect as well as provide for display of the visualization data captured by the probe to a practitioner and a patient in real-time.

6. The apparatus of claim 5, wherein the PPE/AR goggles further comprise a motorized optical lens arrangement that provides a single AR viewing mode which is enabled by optical wave guides built into the main lens enabling simultaneous view of two light sources, or multiple viewing modes to avoid the concerns of motion sickness associated with AR, wherein the multiple viewing modes can switch between alternate birdbath-image projection mode (BIPM) and horizontal-split screen mode (HSSM).

7. The apparatus of claim 1, wherein the probe tip is metal, is constructed in a series of graduated sizes, and removeably attached using one or more probe tip fasteners.

8. The apparatus according to claim 1, wherein the software application interface includes a photogrammetry module for creating multi-dimensional models of the oral cavity.

9. The apparatus according to claim 1, further comprising a hot-swap battery subsystem configured to allow battery replacement without interrupting the operation of the device, and wherein the probe apparatus and PPE/AR goggles 40 integrates global positioning capacity, and incorporates private GNSS, Bluetooth, and Wi-Fi-based tracking features, enabling a single doctor or an office to locate their devices indefinitely, ensuring device security.

10. The apparatus according to claim 1, wherein the probe's software is configured with machine-learning models for automated optical inspection and oral-cavity health classification.

11. The apparatus according to claim 1, further comprising an auxiliary adapter for connecting to a computer, enabling data storage and processing for clinical diagnostics and training purposes.

12. The apparatus according to claim 1 wherein the system further comprises one or more CPUs communicatively connected with embedded software for processing and storing information.

13. The apparatus of claim 5 wherein the PPE/AR goggles further comprise a microphone; and wherein the communication modules and software include speech synthesis and translation language processing components to capture and process microphone data, and are in communication with the microphone, rendering the system capable of translating practitioners' speech for non-English or native-language speaking patients.

14. A method for using the apparatus of claim 1 for intraoral dental examination, the method comprising the steps of:
Device activation: initially turning on the probe and augmented reality (AR) goggles by inputting a charged battery or connecting the device to an outlet, while powering the auxiliary computer adapter; and waking up the device by pressing a capacitive button or utilizing a low-power speech command;
Device pairing: pairing the probe, goggles, and adapter via wireless communication, enabling bidirectional peer-to-peer wireless communication through function-specific wireless transmitters once paired;
Biometric data acquisition: employing a comprehensive sensor suite on the probe, including thermal imagery sensors, visible-light image sensors, biopotential sensors, a load cell sensor, a time-of-flight (tof) sensor, an accelerometer, a magnetometer, a gyroscope, and a microphone to acquire crucial biometric data for assessing the patient's oral health;
Wireless data transmission: transmitting the captured visual and biometric data wirelessly to external devices including the AR/PPE goggles and auxiliary computer adapter, ensuring seamless integration with the practitioner's existing digital infrastructure;
Data processing and display: processing the data using an embedded software application on the CPU of the probe, goggles, and adapter to enhance clinical assessments and decision-making by providing detailed patient health insights;
Multi-dimensional including 3D model generation: utilizing a desktop software application with photogrammetry technology to generate multi-dimensional models of the oral cavity, requiring the probe to wirelessly connect to the auxiliary adapter and initialize in photogrammetry mode to capture a series of still images for comprehensive diagnostic and treatment planning;
Data storage and processing: storing and processing the captured data on a connected computer using internal memory on the probe, goggles, or auxiliary adapter and wi-fi-based cloud storage services, supporting clinical diagnostics and training for practitioner education and patient record management;
Communication of results: sharing patient photos from the probe or goggles via SMS messaging leveraging the internal electronic SIM card, or from cloud storage services; integrating the desktop software application into video-conferencing workflows for teaching and remote practitioner participation; and
Device global positioning: integrating private GNSS, bluetooth, and wi-fi-based tracking features within the probe and goggles to enable device location by a single doctor or an office indefinitely.

15. The method according to claim 14, further comprising the step of determining pocket depth and applied force to a periodontal pocket or gingival sulcus using the sensor suite.

16. The method according to claim 14, further comprising the step of conducting pocket depth estimation through multiple levels of redundancy, including machine-vision analysis of graduated probe tip markers, machine vision depth analysis, and probe-tip electrical resistance-to-depth relationship, supplemented by ToF distance estimation.

17. The method according to claim 14, further comprising generating multi-dimensional models of the oral cavity using photogrammetry software integrated into the software application, displaying the processed data on PPE/AR goggles in real-time to both a dental practitioner and a patient, and storing and processing captured data on a connected computer through an auxiliary adapter for clinical diagnostics and training purposes.

18. The method according to claim 14, further comprising the step of performing automated optical inspection and oral-cavity health classification with embedded machine-learning models.

19. The method according to claim 14, further comprising the step of enabling battery hot-swap during procedures without data loss or operational interruption incorporating battery authentication to prevent tampering, and facilitating on-device or external hub battery charging.

20. A kit for storing and transporting the apparatus of claim 1, including one or more of the apparatus of claim 1, PPE/AR goggles, replacement components including batteries, probes and probe tips, sensors, a video or audiovisual interface, a video or audiovisual monitor adapter, an external data storage drive, and electronic connecting and charging components; all of which are removeably positioned in a durable and protective case.
